(19) United States
(12) Patent Application Publication
(10) Pub. No.: US 2003/0125388 A1
(43) Pub. Date: Jul. 3, 2003

(54) 5-Aminolevulinic acid formulation dissolved/dispersed in non-aqueous solvents

(76) Inventors: Bruno Gander, Immensee (CH); Antje Bunke, Dresden (DE); Gerd Burmeister, Oberarth (CH)

Correspondence Address: SUTHERLAND ASBILL & BRENNAN LLP, 999 PEACHTREE STREET, N.E., ATLANTA, GA, 30309, US

(21) Appl. No.: 10/182,535

(22) PCT Filed: Jan. 29, 2001
(86) PCT No.: PCT/EP01/00930

(30) Foreign Application Priority Data
Jan. 28, 2000 (DE) 100 03 620.1

(51) Int. Cl. A61K 31/195; A61K 31/22

(52) U.S. Cl. 514/561; 514/550

(57) ABSTRACT

The invention concerns compositions that contain 5-aminolevulinic acid or/and derivatives thereof dissolved or dispersed in a non-aqueous liquid. The invention in particular concerns a two-chamber system which contains compositions of 5-aminolevulinic acid and/or derivatives thereof in which the active substances are located in a non-aqueous phase and which are ready-to-use after mixing with an aqueous phase.

The invention concerns compositions which contain 5-aminolevulinic acid or/and derivatives thereof dissolved or dispersed in a non-aqueous liquid. It also concerns a two-chamber system which comprises a non-aqueous 5-aminolevulinic acid formulation in a first chamber and an aqueous carrier system in a second chamber.

Photodynamic therapy is a new and promising method for the treatment of various pre-malignant and malignant diseases which are associated with cell proliferation. The principle of photodynamic therapy is based on the introduction of a so-called photosensitizer into the tumour tissue which is converted into a cytotoxic substance by irradiation with light of a suitable wavelength that ultimately leads to the destruction of the cells. The selectivity of this method is based on the fact that the sensitizer accumulates to a greater degree in rapidly proliferating tumour cells compared to normal tissue. The sensitizer present in the tumour cells can be specifically activated by locally restricted irradiation with light which leads to the destruction of the cancer cells while not significantly affecting the healthy tissue.

Previously a mixture of haematoporphyrin derivatives that can be administered intravenously has usually been used as the photosensitizer. Despite encouraging clinical results in various types of cancer, these haematoporphyrin derivatives have various disadvantages. Firstly, relatively high concentrations of the active substance occur in normal tissue due to the low tumour selectivity and the slow elimination from the body. This results in undesired photochemical reactions in healthy tissue during the irradiation. Secondly, this treatment results in a general light sensitivity and hence the patient cannot be exposed to daylight for a duration of about four weeks.

The high concentrations of active substance in normal tissue and hence the undesired side effects can be reduced in certain cases, especially in dermatological and gynaecological applications, by developing formulations of the active substance that can be applied topically instead of the known systemic formulations. WO 95/05813 for example describes a plaster impregnated with 5-ALA for dermal application. Furthermore attempts have been made to reduce the light sensitivity by using precursors of photosensitizers which are photochemically inactive and are only converted into a photosensitizer within the target cell.

5-Aminolevulinic acid is an endogenous substance which is synthesized from glycine and succinyl-CoA. The highly photoactive protoporphyrin IX is formed as part of haem biosynthesis from 5-aminolevulinic acid (5-ALA) in several reaction steps that proceed rapidly and the photoporphyrin IX is subsequently converted in a slow reaction into haem. A natural control mechanism inhibits the endogenous synthesis of 5-ALA as well as the degradation of protoporphyrin IX when the haem concentration is too high.

This control mechanism is circumvented by exogenously administering synthetic 5-ALA which leads to an increased production of protoporphyrin IX. Since its degradation is still inhibited by the natural control mechanism, protoporphyrin IX accumulates in the cells. Protoporphyrin IX can undergo a photochemical oxidation reaction when it is irradiated with light and hence acts as a photosensitizer. When the sensitizer molecule absorbs a quantum of light it is firstly changed into an electronically excited state (singlet state) which is relatively short-lived and releases its excess energy either within a nanosecond by emitting a fluorescence photon or it is converted into a relatively long-lived triplet state. Energy can be transferred from this triplet state to the oxygen molecules present in the cell. The singlet oxygen which is formed in this process is cytotoxic especially for proliferating cells since it reacts with cell components e.g. the cell membrane and mitochondria or it triggers the formation of radicals which damage the cells. In addition the irradiation of the photosensitizer results in a characteristic fluorescence radiation which can be used for detection reactions for example to detect proliferating cells.

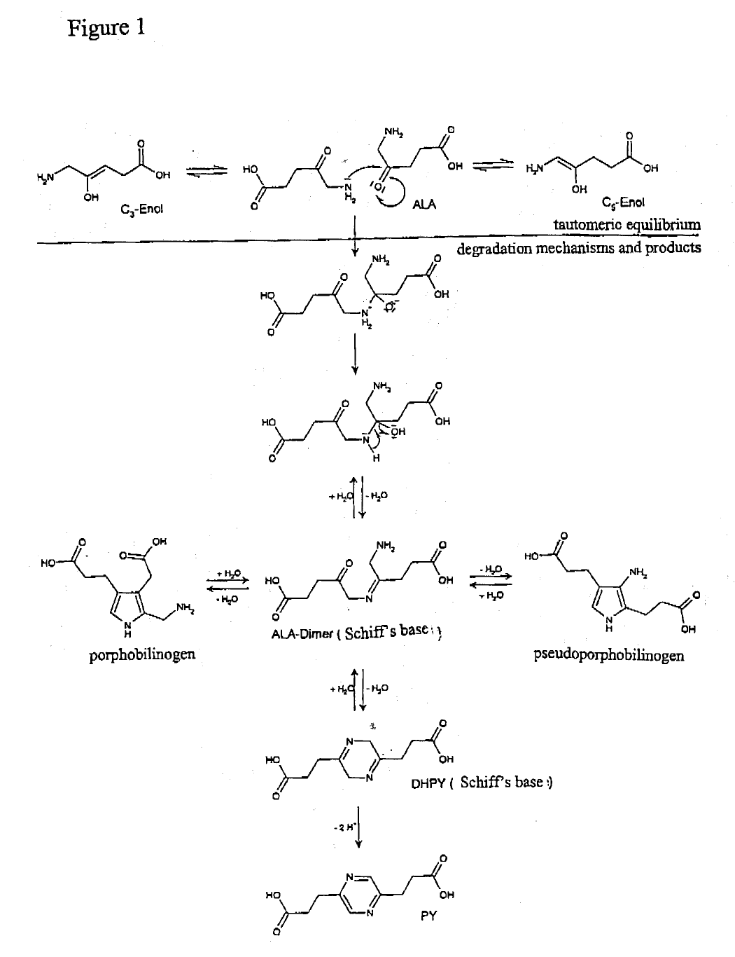

5-ALA is a chemically extremely unstable substance which is subject to a broad spectrum of decomposition reactions (see e.g. Granick and Mauzerall, J. Biol. Chem. 232 (1958), 1119-1140; Franck and Stratmann, Heterocycles 15 (1991), 919-323, Jaffe and Rajagopalan, Bioorg. Chem. 18 (1990), 381-394; Butler and George, Tetrahedron 48 (1992), 7879-7886; Novo et al., J. Photochem. Photobiol. B: Biol. 34 (1996), 143-148; Scott, Biochem. J. 62 (1955), 6P; Dalton et al., Pharm. Res. 16 (1999), 288-295). These decomposition reactions are shown schematically in FIG. 1. As an α-aminoketone, 5-ALA forms a dimeric Schiff's base (DHPY) which is readily oxidized to the aromatic compound PY. It can be converted into porphobilinogen or pseudoporphobilinogen in secondary reactions. The first reaction step of all decomposition stages is the formation of the Schiff's base via the unstable intermediary stages shown in FIG. 1; this is a strongly pH dependent equilibrium in which high pH values, e.g. above pH 5, accelerate the decomposition of 5-ALA. Only an acidic aqueous solution of 5-ALA HCl proves to be adequately stable. However, pH optimization is not a suitable means for stabilizing 5-ALA as a pharmaceutical preparation since a strongly acidic medium cannot be used therapeutically.

In addition to the instability of 5-ALA, its pronounced ionic character is a problem with regard to bioavailability. 5-ALA is present as a zwitterion in the physiologically tolerated pH range (pH 5 to 8) i.e. with a dissociated carboxyl group and a protonated amino group. It is well-known that such charged substances do not readily cross membranes i.e. they are only transported to a slight extent through epithelia and through cell membranes. Thus their bioavailability is low. This also explains the fact that 5-ALA has had to be used in very high doses in previous clinical applications.

Hence the object of the present invention was to provide compositions containing 5-ALA which at least partially eliminate the known disadvantages of the prior art and in particular have an improved chemical stability and an improved membrane permeability.

This object is achieved by introducing 5-ALA or/and derivatives thereof in non-aqueous liquids having a dielectric constant ∈ of <80 at 25° C. and these liquids are preferably physiologically tolerated and miscible with water. Examples of such liquids are 1,2-propylene glycol and glycerol.

Hence one subject matter of the invention is a composition which contains an active substance selected from 5-ALA or/and a derivative thereof dissolved or dispersed in a non-aqueous liquid which has a dielectric constant ∈ of less than 80 at 25° C.

According to the invention the composition contains an active substance selected from 5-aminolevulinic acid or/and a derivative thereof. “Derivative” in particular means salts, esters, complexes and addition compounds. The active substance is particularly preferably 5-aminolevulinic acid or a salt or ester thereof. Preferred examples of salts and esters are 5-ALA hydrochloride, sulphate, nitrate, phosphate, borate, tannate, lactate, glycolate, succinate, citrate, tartrate, embonate and 5-ALA-methylate, ethylate, propionate, butyrate, hexanoate, octoanate, dodecanoate, myristate, palmitate, oleate.

Due to the dissolution or dispersion in non-aqueous liquids, the active substance 5-ALA is preferably at least partially in an enol form which is formed predominantly in liquids that are less polar than water. The presence of the enol form leads to a yellow colouration of the composition which, however, is not due to the decomposition of 5-ALA to one of the compounds shown in FIG. 1. Formation of the enol form stabilizes 5-ALA which retards the formation of the Schiff's base according to FIG. 1 and consequently also delays its reaction to form further degradation products. Furthermore the enol form of 5-ALA which is less polar than the keto form improves its uptake through physiological membranes. Hence this also leads to an improved bioavailability in addition to the chemical stabilization.

Preferred examples of non-aqueous liquids that can be used to dissolve or disperse the active substance are pharmaceutically acceptable solvents such as alcohols e.g. higher alcohols such as C₁-C₂₀ alcohols, ethers and esters, multivalent e.g. divalent or trivalent alcohols and esters thereof e.g. glycerol and its mono, di and triesters with C₁-C₂₀ carboxylic acids, 1,2-propylene glycol, 1,3-propylene glycol and monoesters and diesters thereof with C₁-C₂₀ carboxylic acids, poly(alkylene oxides), in particular poly(ethylene or/and polypropylene oxides) with up to 1000 alkylene units and esters thereof, phospholipids, esters of higher carboxylic acids, sulfoxides such as dimethylsulfoxide (DMSO), N-vinylpyrrolidone and N,N-dimethylacetamide. Mixtures of two or more of the said substances are also suitable. In some embodiments it may be preferable when the non-aqueous liquid is not an alkane diol, alkane triol or an organic acid.

In addition the composition can also contain substances which serve to solidify the 5-ALA formulation, e.g. as a salt or in the form of esters, at a low temperature e.g. refrigerator temperatures, which liquefy again at room temperature or body temperature and thus can contribute to a further increase in the storage stability of 5-ALA. Examples of such substances with temperature-dependent solid-liquid properties are vegetable oils such as cottonseed oil, peanut oil, sesame oil, surfactants such as Cremophor®EL, PEG 400 monostearate, PEG 600-monostearate, polysorbate (Tween 61) and solubilizers such as Solutol® HS15, isopropylmyristate and isopropylpalmitate. Most of these substances are not per se good solvents for 5-ALA but can keep 5-ALA or derivatives thereof in solution in combination with other substances already mentioned e.g. glycerol or propylene glycol and, moreover, give the composition the property of being able to solidify at a low temperature and liquefy again at ambient temperature.

Furthermore the composition can optionally additionally contain water or an aqueous solution, preferably in small amounts of up to a maximum of 50% by weight, particularly preferably up to a maximum of 25% by weight relative to the total weight of the composition. Water is preferably not added until immediately before the intended application of the composition.

The amount of the active substance e.g. 5-ALA in the composition essentially depends on the intended application purpose. Usually about 1 to 25% by weight relative to the total weight of the composition is present. However, higher or lower dosages are feasible. A proportion of 1 to 15% by weight and in particular of about 2 to 10% by weight has proven to be suitable for applications in connection with photodynamic therapy.

If the composition is intended to be a component of a kit as described herein, the concentration of the active substance can be adjusted (e.g. to 1-60% by weight) such that the desired application concentration is obtained after mixing with other kit components.

The composition can also contain auxiliary substances or/and additives and in particular substances which are commonly used in cosmetics or pharmaceuticals. Examples of such substances are buffers, stabilizers, additional emulsifiers, thickeners etc.

Another subject matter of the present invention is a composition according to the invention in the form of a pharmaceutical preparation. In this case the composition is free from components that are not pharmaceutically acceptable and is preferably free from components which are for example irritative. In addition to the already mentioned carrier substances the pharmaceutical preparation can contain other auxiliary substances or/and additives which are acceptable and are preferably well-tolerated.

The composition can be present as a solution, suspension, emulsion, microemulsion, gel, ointment, spray, foam, suppository or ovulum.

The pharmaceutical preparation can be present in a form which is suitable for a systemic administration such as an injectable liquid. However, for dermatological and gynaecological applications the preparation is preferably present in a form that is suitable for a topical application. The preparation has favourable properties for the respective desired form of administration e.g. a suitable viscosity, rheological properties, wetting and penetration capability in order to ensure that an adequate penetration into the target tissue occurs after the administration. These said properties can be adjusted by adding thickeners and wetting agents as well as substances which facilitate penetration such as polyethylene glycol stearyl ethers, polyethylene glycol stearates, polysaccharides such as polysaccharide B-1459, Softisan®378, clofibrinic acid, 2-pyrrolidone, acetyl cysteine or/and carbocysteine.

In addition to 5-ALA or derivatives thereof, the composition can also contain additional drugs which are for example selected from local anaesthetics, antibiotics, prostaglandins, steroidal and non-steroidal anti-inflammatory agents, growth hormones, cytokines such as TNF, sexual hormones or vitamins.

In order to produce the composition or pharmaceutical preparation according to the invention the active substance is dissolved or dispersed in the non-aqueous liquid. Additives that may be optionally present can be added before, during or/and after the dissolution or dispersion.

The process is preferably carried out in the absence of air for example by applying a vacuum or/and a protective gas atmosphere. Furthermore it is preferable to carry out the process in the absence of light. The process is carried out at a temperature at which the formation of the desired composition can occur and at which the components and in particular the active substance are adequately stable. In general a temperature range of about 5 to 45° C. has proven to be suitable. For a pharmaceutical application it is ensured that the resulting product is sterile e.g. by using sterile starting materials and maintaining sterile process conditions or/and by a sterilization step after the production.

Another subject matter of the present invention is a kit which comprises a composition containing 5-aminolevulinic acid (5-ALA) or/and a derivative thereof as described above and, separate therefrom, a composition containing water in particular an aqueous buffer system, an aqueous solution or an aqueous carrier system.

The kit according to the invention preferably contains, as a component, a non-aqueous 5-ALA formulation in particular a 5-ALA formulation having a water content in % by weight of <2%, more preferably <1% and particularly preferably <0.1%. The formulation of the kit containing 5-ALA is most preferably completely free from water.

Hence the kit according to the invention contains a first component a) which is a composition in which it is possible to store 5-ALA or derivatives thereof dissolved or dispersed in a non-aqueous liquid with a high stability. Separate therefrom the second component b) of the kit comprises an aqueous system in particular an aqueous buffer system, an aqueous solution or an aqueous carrier system. Component b) of the kit preferably contains at least 50% by weight water, more preferably at least 80% by weight water and most preferably at least 90% by weight water. The component b) can also be pure water. After mixing the two liquids which are present separately in the kit according to the invention, a mixture composition is obtained which has advantageous properties for the application while at the same time achieving a high long-term stability of the individual components by the arrangement in a kit.

In particular the following advantages are achieved by the separate provision of 5-aminolevulinic acid or/and a derivative thereof in a non-aqueous liquid and an aqueous system:

In the non-aqueous phase of component a) the active substance 5-ALA or/and a derivative thereof is mainly present in the enol form and can thus not convert into the dimeric dihydroxypyrazine derivative. A keto form of the 5-aminolevulinic acid is a prerequisite for a reaction with itself to form a Schiff's base resulting in the dihydroxypyrazine derivative. Whereas 5-ALA is mainly present in the keto form in an aqueous solution, it is mainly present in the enol form in the non-aqueous phase of component a) of the kit according to the invention and hence this undesired reaction cannot occur. Thus the stability of the active substance in the non-aqueous system that is present in component a) of the kit according to the invention is considerably increased. Thus the non-aqueous composition is suitable as a storage form for 5-ALA with a high long-term stability.

If a non-aqueous formulation, such as that contained in the kit according to the invention as component a), is directly applied to tissue, this can result in a very low pH when it comes into contact with tissue water which decreases the bioavailability of 5-ALA. The kinetics of protoporphyrin IX (PPIX) synthesis is considerably increased at neutral or slightly basic pH value compared to acidic ambient conditions. Furthermore direct application can result in undesired cell damage in the healthy tissue due to the low pH. Hence this could result in the loss of some of the selectivity of the photodynamic therapy (PDT) which is used especially when the purpose is to selectively damage or destroy mutated, i.e. unhealthy, cells.

Component b) of the kit according to the invention is used to adjust the pH and hence increases the bioavailability of the active substance and the selectivity of cell damage in a photodynamic therapy.

Consequently component b) preferably contains an aqueous solution which is suitable for setting a physiological pH when components a) and b) are mixed. The aqueous system of component b) preferably has a pH of >7, more preferably a pH of >8 and most preferably a pH of >9. The composition of component b) containing water is preferably an aqueous solution and in particular an alkaline solution, an aqueous buffer system or/and an aqueous carrier system which contains auxiliary substances such as substances which assist the application in addition to water. The composition containing water is preferably an NaOH or KOH solution or a phosphate or carbonate buffer preferably with a suitable capacity to adjust the pH to a neutral or slightly alkaline range after combination with component a). The pH is preferably set to a pH of ≧5, more preferably to ≧6, even more preferably to ≧7 and most preferably to ≧8.

The kit according to the invention is particularly preferably intended for a topical application. The two kit components are preferably combined shortly before the application, for example 10 sec to 1 h before the application, but can also take place during the application.

The kit according to the invention can optimize the storage properties as well as the application properties of a composition containing 5-ALA as the active substance.

The kit according to the invention is preferably present in the form of a two-chamber system in which component a) is located in the first chamber of the system and component b) is located in the second chamber of the system. Shortly before the application or during the application the contents of the two chambers are mixed in order to set a pH in the neutral or slightly alkaline range.

Thus the invention concerns in particular a 2-chamber system which contains compositions of 5-aminolevulinic acid and/or derivatives thereof in which the active substances are stored in a non-aqueous phase and which are used after mixing with an aqueous phase.

An important field of application for the compositions and kits according to the invention is the field of photodynamic therapy, in which case the composition is particularly preferably applied topically. Furthermore the composition according to the invention can also be used for all diseases whose treatment comprises inhibiting the proliferation of cells or tissue or killing cells or tissue by photoactivating a sensitizer formed from 5-ALA. These include in particular diseases that are associated with an increased cell proliferation since in this case a particularly high degree of enrichment of the photosensitizer occurs due to the increased cell metabolism in the diseased cells.

The composition and kits according to the invention are hence suitable for the treatment of tumour diseases such as basal cell carcinomas, squamous-cell carcinomas, morbus bowen, actinic keratosis, condylomata acuminata (CIN), epithelial neoplasia of the vulva (VIN), nodular and subcutaneous cancer diseases. An example of a non-tumourous disease is psoriasis or acne.

The treatment is carried out for example by a topical application of a composition containing the active substance e.g. 5-ALA and subsequent incubation in order to allow an adequate amount of 5-ALA to penetrate into the tissue to be treated. During the incubation, the treated site is protected from light irradiation for example by covering it in order to prevent an undesired premature activation. After the incubation period which is generally about 1 to 8 h and usually about 4 h, the tissue is irradiated with a light source with an adequate dose of radiation. Suitable light sources include lamps which radiate white light and monochromatic light sources such as a laser and in particular argon dye lasers with an emission at about 630 nm. The radiation dosages are usually in a range of about 20 J/cm² up to several 100 J/cm² per application.

Another field of application for the compositions according to the invention concerns the detection of the presence of proliferating cells in a specimen e.g. a tissue specimen. The detection is based on a selective enrichment of a photosensitizer generated by metabolism of the active substance in the proliferating cells compared to normal cells. The active substance is preferably 5-ALA and the photosensitizer is preferably protoporphyrin IX. The enrichment of the photosensitizer can be determined by photodiagnostic methods e.g. by irradiating with light having a wavelength of 405 nm and measuring the fluorescence radiation generated by the photosensitizer. The compositions according to the invention are particularly suitable for use in tumour diagnostics.

Another subject matter of the invention is the use of the composition according to the invention or/and of the kit according to the invention to produce a medicament for photodynamic therapy.

Finally the invention concerns another kit which contains a composition according to the invention suitable for topical application or for application in body cavities and one or more auxiliary agents. Such auxiliary agents are for example a cover material such as a plastic foil which for example in the case of a topical application but not in the case of an application in body cavities, is applied to the site to be treated after the composition has been applied to prevent a premature activation by light, means for attaching the cover material or means for applying the composition to the site to be treated.

The following figures and examples are intended to further elucidate the invention.

FIG. 1: shows a schematic representation of decomposition reactions of 5-aminolevulinic acid (5-ALA).

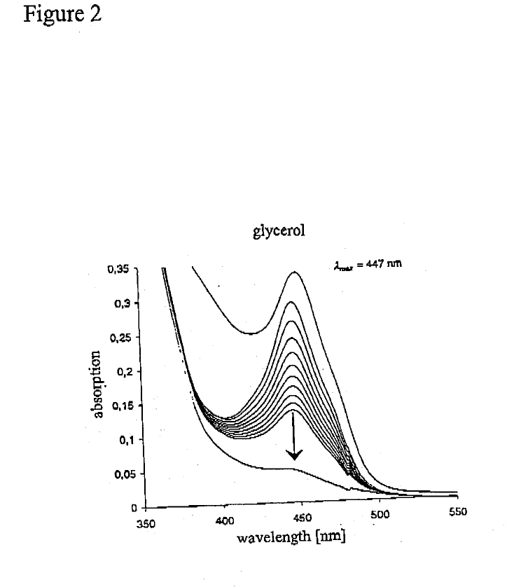

FIG. 2: shows the time-dependent change of the UV-VIS spectrum of a 10% 5-ALA solution in anhydrous glycerol (undiluted and after dilution with water in a ratio of 1:1 recorded at intervals of 0.2 min).

### EXAMPLES

1. Preparation of Non-aqueous 5-ALA Compositions

10% solutions (weight/vol) of 5-ALA in 1,2-propylene glycol and glycerol were prepared. After complete dissolution a yellow colouration was found but which was not due to decomposition of 5-ALA into one of the degradation products listed in FIG. 1. Hence neither DHPY, PY nor porphobilinogen were detected in capillary electrophoresis.

Hence the colouration of the solution was due to the formation of the enol form of 5-ALA. This was confirmed by UV-VIS measurements. An absorption band at 447 nm was found in glycerol as well as in 1,2-propylene glycol which was the cause of the optically detectable yellow colour. This spectral shift is due to the enolization of 5-ALA which has already been observed in aqueous alkaline solutions (Monteiro et al., Arch, Biochem. Biophys. 271 (1989), 206-217).

If water is added in a ratio of 1:1 to the 10% water-free 5-ALA solution, one observes a disappearance of the yellow colour of the solution within a few minutes. This was detected by measuring the decrease of absorbance at 447 nm (FIG. 2). In a subsequent solution diluted 1:100 with water, a UV spectrum of 5-ALA was observed without the presence of by-products.

1. Composition, characterized in that
it contains an active substance selected from 5-aminolevulinic acid (5-ALA) or/and a derivative thereof dissolved or dispersed in a non-aqueous liquid which has a dielectric constant ∈ of less than 80 at 25° C. which acts as stabilizer of 5-aminolevulinic acid (5-ALA) or/and the derivative thereof.

2. Composition as claimed in claim 1, characterized in that
it is free from water.

3. Composition as claimed in claim 1 or 2, characterized in that
the derivatives are selected from salts and esters of 5-ALA.

4. Composition as claimed in one of the claims 1 to 3, characterized in that
the active substance is mainly present in the enol form.

5. Composition as claimed in one of the previous claims, characterized in that
the non-aqueous liquid is selected from alcohols, ethers, esters, poly(alkylene glycols), phospholipids, DMSO, N-vinylpyrrolidone, N-N-dimethyl acetamide and mixtures thereof.

6. Composition as claimed in one of the previous claims, characterized in that
the non-aqueous liquid is at least partially miscible with water.

7. Composition as claimed in one of the previous claims, characterized in that
it additionally contains water.

8. Composition as claimed in one of the previous claims, characterized in that
the active substance is present in an amount of 1 to 25% by weight in particular of 1 to 15% by weight relative to the total weight of the composition.

9. Composition as claimed in one of the previous claims, characterized in that
it additionally contains auxiliary substances or/and additives that are commonly used in cosmetics or pharmaceuticals.

10. Composition as claimed in one of the previous claims, characterized in that
it is present as a solution, suspension, emulsion, microemulsion, gel, ointment, spray, foam, suppository or ovulum.

11. Composition as claimed in one of the previous claims, characterized in that
it is in the form of a pharmaceutical preparation.

12. Composition as claimed in claim 11, characterized in that
it can be applied topically or systemically.

13. Composition as claimed in one of the previous claims, characterized in that
it contains an additional medicinal substance.

14. Kit, in particular for therapeutic or diagnostic use, comprising
a) a composition containing 5-aminolevulinic acid (5-ALA) or/and a derivative thereof as claimed in one of the claims 1 to 13 and
b) a composition containing water.

15. Kit as claimed in claim 14, characterized in that
component a) is free from water.

16. Kit as claimed in one of the claims 14 or 15, characterized in that
component b) contains an aqueous solution which is suitable for adjusting a physiological pH in the mixture of components a) and b).

17. Kit as claimed in one of the claims 14 to 16, characterized in that
component b) contains an NaOH or KOH solution or/and a phosphate or/and carbonate buffer.

18. Kit as claimed in one of the claims 14 to 17, characterized in that
it is present in the form of a double-chamber system in which component a) is located in the first chamber of the system and component b) is located in a second chamber of the system.

19. Use of a composition containing an active substance selected from 5-ALA or a derivative thereof as claimed in one of the claims 1 to 13 or a kit as claimed in one of the claims 14 to 18 for the production of a pharmaceutical preparation for the treatment or diagnosis of diseases associated with cell proliferation.

20. Use as claimed in claim 19, characterized in that
the composition is applied topically or systemically.

21. Use as claimed in claim 19 or 20 for the treatment or diagnosis of tumour diseases.

22. Use as claimed in one of the claims 19 to 21 for a photodynamic therapy.

23. Use as claimed in claim 21 or 22, characterized in that
the disease is a basal cell carcinoma, squamous-cell carcinoma, morbus bowen, actinic keratosis, condylomata acuminata (CIN), intraepithelial neoplasia of the vulva (VIN), or a nodular or subcutaneous cancer disease.

24. Use as claimed in claim 23, characterized in that
the disease is psoriasis or acne.

25. Use of a composition as claimed in one of the claims 1 to 13 or of a kit as claimed in one of the claims 14 to 18 for producing a diagnostic test or medicament for photodynamic therapy.

26. Kit comprising a composition that can be applied topically as claimed in claim 12 and at least one component selected from
(a) an essentially light-impermeable sheet-like material,
(b) means for attaching the sheet-like material to a site of application and
(c) means for applying the composition to a site of application.

27. Kit as claimed in claim 26, additionally comprising
(d) an aqueous buffer system.

28. Use of a non-aqueous liquid which has a dielectric constant ∈ of less than 80 at 25° C. to stabilize 5-aminolevulinic acid (5-ALA) or/and a derivative thereof, wherein the 5-aminolevulinic acid or/and the derivative thereof is dissolved or dispersed in the non-aqueous liquid.
